SEQUENCE LISTING (14 sequences)

SEQ ID NO: 1 - DNA, 5518 bp, Artificial
  recombinant plasmid p-LC
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagagtcgacaaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgcctatgagtgtgctcactcaggtcctggcgttgctgctgctgtggcttacaggtacgcgttgtgacatcgtgatgacccagtctccatcctccctgtctgcatctgtaggagacagagtcaccatcacttgccgggcaagtcagaacattatccgctatttaaattggtatcagcagaagccagggaaagcccctaggctcctgatctatggtgcgtccactttgcaaagtggggtcccatcaaggttcagtggcagtggatctgggacagatttcactctcaccatcagtagtctgcaacctgaagattttgcaacttactactgtcaacagagttaccgtacccctccattcactttcggccctgggaccaagcttgaaatcaaacctgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttagaagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagattctagagtcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 2 - protein, 234 aa, Artificial
  Synthetic Construct
MSVLTQVLALLLLWLTGTRCDIVMTQSPSSLSASVGDRVTITCRASQNIIRYLNWYQQKPGKAPRLLIYGASTLQSGVPSRFSGSGSGTDFTLTISSLQPEDFATYYCQQSYRTPPFTFGPGTKLEIKPVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - DNA, 6238 bp, Artificial
  recombinant plasmid p-HC
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagagtcgacaaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggcttgggtgtggaccttgccattcctgatggcagctgcccaaggtgtcgacgcacaggtgaaactgctcgagtctgggggaggcgtggtccagccgggggggtccctgagactctcctgtgaagcgtctggattcgccctcagaagttctggcatgcactgggtccgccaggctcctggcaaggggctggagtgggtggcacttatatggtttgatggaagtatcagatcgtatgcagaatccgtgaagggccgattcaccatctccagagacacttccaagaacaccctatatctccaaatgcgcagtctgagtgccgacgacacggctgtgtattactgtgcgagagacaaggcggttcggggaattagcaggtacaactattacatggacgtctggggcaaagggaccacggtcaccgtctcctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatgataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagattctagagtcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 4 - protein, 474 aa, Artificial
  Synthetic Construct
MAWVWTLPFLMAAAQGVDAQVKLLESGGGVVQPGGSLRLSCEASGFALRSSGMHWVRQAPGKGLEWVALIWFDGSIRSYAESVKGRFTISRDTSKNTLYLQMRSLSADDTAVYYCARDKAVRGISRYNYYMDVWGKGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 5291 bp, Artificial
  recombinant plasmid p-p18
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggccgagccttgggggaacgagttggcgtccgcagctgccaggggggacctagagcaacttactagtttgttgcaaaataatgtaaacgtcaatgcacaaaatggatttggaaggactgcgctgcaggttatgaaacttggaaatcccgagattgccaggagactgctacttagaggtgctaatcccgatttgaaagaccgaactggtttcgctgtcattcatgatgcggccagagcaggtttcctggacactttacagactttgctggagtttcaagctgatgttaacatcgaggataatgaagggaacctgcccttgcacttggctgccaaagaaggccacctccgggtggtggagttcctggtgaagcacatggccagcaatgtggggcatcggaaccataagggggacaccgcctgtgatttggccaggctctatgggaggaatgaggttgttagcctgatgcaggcaaacggggctgggggagccacaaatcttcaataataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 6 - protein, 168 aa, Artificial
  Synthetic Construct
MAEPWGNELASAAARGDLEQLTSLLQNNVNVNAQNGFGRTALQVMKLGNPEIARRLLLRGANPDLKDRTGFAVIHDAARAGFLDTLQTLLEFQADVNIEDNEGNLPLHLAAKEGHLRVVEFLVKHMASNVGHRNHKGDTACDLARLYGRNEVVSLMQANGAGGATNLQ

SEQ ID NO: 7 - DNA, 5279 bp, Artificial
  recombinant plasmid p-p21
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatgtcagaaccggctggggatgtccgtcagaacccatgcggcagcaaggcctgccgccgcctcttcggcccagtggacagcgagcagctgagacgcgactgtgatgcgctaatggcgggctgcatccaggaggcccgtgagcgatggaacttcgactttgtcaccgagacaccactggagggtgacttcgcctgggagcgtgtgcggggccttggcctgcccaagctctaccttcccacggggccccggcgaggccgggatgagttgggaggaggcaggcggcctggcacctcacctgctctgctgcaggggacagcagaggaagaccatgtggacctgtcactgtcttgtacccttgtgcctcgctcaggggagcaggctgaagggtccccaggtggacctggagactctcagggtcgaaaacggcggcagaccagcatgacagatttctaccactccaaacgccggctgatcttctccaagaggaagccctaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 8 - protein, 164 aa, Artificial [sequence omitted]

SEQ ID NO: 9 - DNA, 5252 bp, Artificial
  recombinant plasmid p-aFGF
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggctgaaggggaaatcaccaccttcacagccctgaccgagaagtttaatctgcctccagggaattacaagaagcccaaactcctctactgtagcaacgggggccacttcctgaggatccttccggatggcacagtggatgggacaagggacaggagcgaccagcacattcagctgcagctcagtgcggaaagcgtgggggaggtgtatataaagagtaccgagactggccagtacttggccatggacaccgacgggcttttatacggctcacagacaccaaatgaggaatgtttgttcctggaaaggctggaggagaaccattacaacacctatatatccaagaagcatgcagagaagaattggtttgttggcctcaagaagaatgggagctgcaaacgcggtcctcggactcactatggccagaaagcaatcttgtttctccccctgccagtctcttctgattaataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 10 - protein, 155 aa, Artificial
  Synthetic Construct
MAEGEITTFTALTEKFNLPPGNYKKPKLLYCSNGGHFLRILPDGTVDGTRDRSDQHIQLQLSAESVGEVYIKSTETGQYLAMDTDGLLYGSQTPNEECLFLERLEENHYNTYISKKHAEKNWFVGLKKNGSCKRGPRTHYGQKAILFLPLPVSSD

SEQ ID NO: 11 - DNA, 6196 bp, Artificial
  recombinant plasmid p-Enbrel
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagagtcgacaaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaactcgacgttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatgataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagattctagagtcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 12 - protein, 460 aa, Artificial
  Synthetic Construct
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQLDVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 5384 bp, Artificial
  recombinant plasmid p-puro
agcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccagatttaattaaggctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggaagatcggaattcgcccttaagctagctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccaggcggccgccatgaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgataagcttggatccaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgagatctgcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggactcgagttaagggcgaattcccgattaggatcttcctagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagccttaattaacctaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccccgatagacggtttttcgccctttgacgctggagttcacgttcctcaatagtggactcttgttccaaactggaacaacactcaaccctatctcggtctattcttttgatttataagggatttttccgatttcggcctattggttaaaaaatgagctgatttaacaaaaatttaacgcgaattttaacaaaatattaacgtttataatttcaggtggcatctttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaatagtggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagtaatggtaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcggttttgctcacatgttctttcctgcgttatcccctgattctgtggataaccgtattaccgcctttgagtgagctgataccgctcgccgcagccgaacgaccgagcgcagcgagtcagtgagcgaggaagcggaag

SEQ ID NO: 14 - protein, 199 aa, Artificial
  Synthetic Construct
MTEYKPTVRLATRDDVPRAVRTLAAAFADYPATRHTVDPDRHIERVTELQELFLTRVGLDIGKVWVADDGAAVAVWTTPESVEAGAVFAEIGPRMAELSGSRLAAQQQMEGLLAPHRPKEPAWFLATVGVSPDHQGKGLGSAVVLPGVEAAERAGVPAFLETSAPRNLPFYERLGFTVTADVEVPEGPRTWCMTRKPGA

(19) United States
(12) Patent Application Publication
(10) Pub. No.: US 2009/0023186 A1
(43) Pub. Date: Jan. 22, 2009

(54) USE OF VALPROIC ACID FOR ENHANCING PRODUCTION OF RECOMBINANT PROTEINS IN MAMMALIAN CELLS

(75) Inventors: Markus Hildinger, Pforzheim (DE); Gaurav Backliwal, Lausanne (CH); Florian Wurm, Lausanne (CH)

Correspondence Address: MARKUS HILDINGER, CRANACHWEG 8, PFORZHEIM, 75173, DE

(73) Assignee: EXCELLGENE SA, Monthey (CH)

(21) Appl. No.: 11/781,252

(22) Filed: Jul. 22, 2007

(51) Int. Cl. C12P 21/00

(52) U.S. Cl. 435/70.3

(57) ABSTRACT

Culturing cells for the commercial production of proteins for diagnosis and therapy is a costly and time consuming process. The equipment required is expensive, and production cost are high. In order to provide commercially viable processes it is desirable to use cell lines which produce large quantities of product with each production run. However, most cells do not produce large quantities of desired product per se either because they do not produce a large quantity of product per unit of time (specific productivity) or because they do not survive long enough in the culture medium (time). Here, we identified that addition of a valproic acid compound to the culture medium increases overall (batch) yield and titer. More importantly, compared to the widely used sodium butyrate, batch yields using a valproic acid compound as a medium additive are significantly higher.

### INCORPORATION-BY-REFERENCE OF MATERIAL SUBMITTED ON A COMPACT DISC

### Reference to Sequence Listing

A paper copy of the Sequence Listing and a computer readable form (CRF) of the sequence listing, containing the file named “VPA.ST25.txt” which is 86 kilobytes in size, and which was created on Jul. 1, 2007 and last modified on Jul. 1, 2007, are herein incorporated by reference. The symbols and format used for nucleotide and amino acid sequence data comply with the rules set forth in 37 C.F.R. section 1.822.

### BACKGROUND OF THE INVENTION

It must be noted that as used herein and in the appended claims, the singular forms “a” and “the” include plural referents unless the context clearly dictates otherwise. Thus, for example, reference to “a cell” or “the cell” includes a plurality (“cells” or “the cells”), and so forth. Moreover, the word “or” can either be exclusive in nature (i.e., either A or B, but not A and B together), or inclusive in nature (A or B, including A alone, B alone, but also A and B together). One of skill in the art will realize which interpretation is the most appropriate unless it is detailed by reference in the text as “either A or B” (exclusive “or”) or “and/or” (inclusive “or”).

A portion of the disclosure of this patent document contains material which is subject to copyright protection. The copyright owner has no objection to the facsimile reproduction by anyone of the patent document or the patent disclosure as it appears in the Patent and Trademark Office file or records, but otherwise reserves all copyright rights whatsoever. The patent owners can be contacted at hildinger@gmx.net.

(1) Field of the Invention

This invention relates to a process for the production of a protein of interest by cell culture, where cells which produce the protein of interest are cultured in the presence of a valproic acid compound, where the valproic acid compound increases batch yield and batch titer—all else equal.

Culturing cells for the commercial production of proteins for diagnosis and therapy is a costly and time consuming process. The equipment required is expensive, and production cost are high. In order to provide commercially viable processes it is desirable to use cell lines which produce large quantities of product with each production run. However, most cells do not produce large quantities of desired product per se either because they do not produce a large quantity of product per unit of time (specific productivity) or because they do not survive long enough in the culture medium (time). Here, we identified that addition of a valproic acid compound to the culture medium increases batch yield and titer—all else equal.

(2) Description of Related Art

A number of chemical compounds are known which are able to act as enhancing agents of protein production in cell lines. Examples of those compounds are DMSO, urea derivatives and alkanoic acids or salts thereof. From amongst such enhancing agents, sodium butyrate has been the subject of intensive study in recent years. This compound has been added to cultures of a variety of naturally occurring and selected cell lines and has been shown to produce many morphological and biochemical modifications. At the molecular level, butyrate is believed to cause hyperacetylation of histones by inhibiting histone deacetylase (histone deacetylase inhibitor; HDAC inhibitor). Generally, butyrate appears to modify gene expression, and in almost all cases its addition to cells in culture appears to arrest cell growth.

Several patents have been filed around the use of alkanoic acids in general and sodium butyrate in particular in the context of enhanced production of a protein of interest, e.g., U.S. Pat. Nos. 5,681,718, 6,117,652, 6,740,505 to name a few. However, the use of a valproic acid compound in the context of enhancing production of a protein has not yet been published in prior art.

Valproic acid is indicated for the treatment of epilepsy (anticonvulsant) and is commercially available under the brand names Depakene, Valproate, Valrelease. It is used as a sole or adjunctive therapy in the treatment of simple or complex absence seizures, including petit mal, and is useful in primary generalized seizures with tonic-clonic manifestations. Valproic acid may also be used adjunctively in patients with multiple seizure types which include either absence or tonic-clonic seizures. Valproic acid also has efficacy as a mood stabilizer in bipolar disorder.

### BRIEF SUMMARY OF THE INVENTION

(1) Substance or General Idea of the Claimed Invention

Context: In a first aspect the present invention provides a process for the production of a protein of interest which comprises culturing cells which produce said protein in the presence of a valproic acid compound, which enhances protein production, wherein the valproic acid compound is present at a concentration at which production of said protein is enhanced.

In a further aspect, the invention provides a process for obtaining a protein by cell culture which comprises the steps of (1) culturing cells which produce said protein in the presence of a valproic acid compound, which enhances protein production, wherein the valproic acid compound is present at a concentration at which production of said protein is enhanced, (2) continuing said culture until said protein accumulates, and optionally (3) isolating said protein.

In yet another aspect, the present invention teaches that production of a protein in the presence of a valproic acid compound is enhanced compared to production of the same protein in the absence of a valproic acid compound.

In yet another aspect, the present invention teaches that production of a protein in the presence of a valproic acid compound is enhanced compared to production of the same protein in presence of butyric acid or sodium butyrate—assuming that the process conditions in both instances are optimized for the corresponding substances.

Nature of the valproic acid compound. The valproic acid compound can be valproic acid, a salt of valproic acid, or a combination of valproic acid and salt(s) of valproic acid.

Preferably, the valproic acid compound is valproic acid or a salt thereof, in particular an alkali metal salt, e.g. sodium salt.

Concentration of the valproic acid compound. The valproic acid compound may be present in the culture medium at a concentration of between 0.002 mmol/l and 200 mmol/l, preferably between 0.01 mmol/l and 50 mmol/l and most preferably between 0.05 mM and 12 mmol/l. In case the cell is a HEK 293 cell, the preferred final concentration of the valproic acid compound is between 0.1 mmol/and 12 mmol/l and the most preferred final concentration of the valproic acid compound is 3.8 mmol/l. In case the cell is a CHO cell, the preferred final concentration of the valproic acid compound is between 0.05 mmol/and 5 mmol/l and the most preferred final concentration of the valproic acid compound is 0.5 mmol/l.

It will be appreciated, however, that the concentration of the valproic acid compound employed may be varied depending on the particular cell line being cultured. The most appropriate concentration of a valproic acid compound for any particular cell line may need to be determined by appropriate small scale tests beforehand in accordance with conventional practice—without undue effort.

Cell density at time of valproic acid compound addition. In another aspect of the present invention, the valproic acid compound can be added at different cell densities such as at least 1 million cells per ml, preferably at least 2 million cells per ml and most preferably at least 4 million cells per ml. The exact cell density at which the valproic acid compound should be added depends on several factors:

(1) The valproic acid compound concentration: The higher the cell density, the higher the final concentration of valproic acid.
(2) The medium: Not all media are able to sustain cell viability above certain cell densities.
(3) The cell line: Different cell lines have different requirements in terms of cell density for cultivation.

All else equal, a higher cell density should be preferred as higher cell densities at a given specific productivity will result in higher batch titers. The most appropriate cell density at the time of valproic acid addition for any particular cell line or process may need to be determined by appropriate small scale tests beforehand in accordance with conventional practice—without undue effort.

Additional compounds: In some embodiments, additional compounds are added in combination with the valproic acid compound where said additional compounds act synergistically with the valproic acid compound. Such additional compounds are—without limitation—IDMTs (inhibitors of DNA methyltransferases) such as azacytidine, RG108, decitabine.

Temperature: In yet another aspect of the present invention, the mammalian cells are cultivated at a temperature of 37 degrees Celsius in the presence of a valproic acid compound, where the concentration of the valproic acid compound is sufficient to enhance protein production. In some embodiments, cells are cultivated at temperatures below 37 degrees Celsius, in a range of 28 degrees to 33 degrees Celsius and preferably at 31 degrees Celsius or 32 degrees Celsius. It has been shown in the literature that lowering the cultivation temperature can result in higher batch yields [7]. This is also applicable to processes where a valproic acid compound is present in the cultivation medium.

Frequency of valproic acid compound addition to the cells: In some embodiments, the valproic acid compound is added only once to the cells; in other embodiments, the valproic acid compound is added more than once to the cells, for example every other day.

Timing of valproic acid compound addition to the cells: In the process according to the invention, the valproic acid compound may be added to the culture medium at, before or after addition of the cells to the culture medium. If desired more than one addition of the valproic acid compound may be employed. Thus, for example, it may be desirable to add the valproic acid compound at the beginning of the culture and then to add more valproic acid compound as the culture proceeds, providing of course that the addition is closely controlled such that the concentration of the valproic acid compound does not go beyond that which is likely to reduce the cell growth rate or cell viability. Typically in this aspect of the invention the cells are grown in culture to provide the levels of biomass required for efficient protein production. The valproic acid compound may be included in the growth medium if desired but in some circumstances, however, it may be desirable to grow the cells in the absence of a valproic acid compound, for instance, when production of the protein has a deleterious effect on the cells. Thus cells may be grown to densities at or approaching maximum cell density in the case of suspension cultures, or to or approaching confluence in the case of adherent cell lines, at which stage they may be transferred to a production medium and the valproic acid compound added at a concentration which enhances protein production but which does not significantly reduce cell viability.

The valproic acid compound may also be used to increase protein production in cells which have stopped growing exponentially and are in stationary or decline phases of growth. This is particularly advantageous when the desired protein is only produced by the cell when it is in these last two phases. Thus in another aspect the invention provides a process for the production of a protein which comprises maintaining cells which produce said protein in culture in the presence of a valproic acid compound which enhances protein production wherein the valproic acid compound is present at a concentration at which production of said protein is enhanced but which does not significantly reduce cell viability (e.g. is substantially non-toxic to the cells).

In yet another aspect, the invention provides a process for the production of a protein which comprises a first stage in which cells which produce said protein, are grown in growth medium until a predetermined cell density has been obtained followed by a second stage in which said cells are maintained in the presence of a valproic acid compound which enhances protein production wherein said valproic acid compound is present at a concentration at which production of said protein is enhanced but at which cell growth is inhibited without significantly reducing cell viability. In said second stage, the cells may still be maintained in the growth medium, where valproic acid has been added to the growth medium (transforming the growth medium into a production medium), or the growth medium might be exchanged with a production medium comprising a valproic acid compound, or the growth medium might be modified, e.g., by adding additional compounds—including a valproic acid compound—to become a production medium.

The growth medium may contain a valproic acid compound which enhances production of the protein though preferably does not. The predetermined cell density to which the cells are grown in the first stage is preferably a cell density suitable for efficient protein production such as a density approaching maximum cell density for the culture or approaching confluence. The concentration of a valproic acid compound used in the second state will generally be as described previously but in particular will be such to arrest cell growth, prolong the period of cell viability and yield an increased level of overall protein production.

In the context of transient gene expression, where the gene expression vector encoding the protein of interest is introduced into the cell by means of transfection, the valproic acid compound is added within 48 hours of transfection and more preferably within 24 hours after transfection. In that instance, the cells are expanded in growth medium prior to transfection, transferred into transfection medium for the transfection, and then transferred into production medium for protein production. In such a setting, only the production medium might comprise the valproic acid compound. Yet, in some embodiments, also the transfection medium might comprise a valproic acid compound.

Stable cell line vs. transient gene expression: In another aspect, the cell which produces the protein of interest has the corresponding transgene stably integrated in its genome, i.e., the cell is a stable producer cell. In yet another aspect, the expression cassette encoding the genetic information for the protein of interest is introduced into the cell by means of transfection. For example, the cells to be transfected will be expanded in a first stage in growth medium, transfected with a gene expression vector encoding the protein of interest in a second stage in transfection medium and cultivated in production medium after transfection for 1 day to 3 weeks or longer. The valproic acid compound might be added to the growth, transfection and production medium, preferably only to the transfection and production medium and most preferably to the production medium only. Transfection methods have been described in detail in prior art (see also the definition section). Most preferably, transfection is performed with 25-kd linear polyethyleneimine.

Cell type: The cell which is used for the production of the protein of interest, be it by means of stable integration or by transient gene expression, can be any mammalian cell, preferably myeloma B (NS0) cells, Per.C6 cells, Chinese Hamster Ovary (CHO) cells, Baby Hamster Kidney (BHK) cells or Human Embryonic Kidney (HEK 293) cells, most preferably CHO or HEK 293 cells.

This embodiment of the invention is particularly applicable to cell lines which are intermediate or low producers of a desired protein, making it possible to substantially increase protein production by use of a valproic acid compound at an appropriate concentration.

Nature of the protein produced: The protein produced by the process is preferably an immunoglobulin or an FC-fusion protein. However, any other protein of interest can be produced by the teachings of the present invention—including, but not limited to, growth factors, hormones, enzymes, enzyme inhibitors, cytokines, lymphokines, interleukins, blood clotting factors, fusion proteins.

Any suitable culture procedure and culture medium may be used to culture the cells in the process of the invention. Suitable culture procedures are well known and understood by workers in the cell culture art. Both serum supplemented and serum free media may be used. Batch and continuous fermentation procedures, suspension and adherent, e.g. microcarrier culture methods and stirred tank and airlift fermenters may be used as appropriate having regard to cell type.

The production of the protein during the culture may be monitored by general assay techniques such as enzyme linked immunosorbent assay or immunoradiometric assay adapted for the particular protein in question.

The desired protein may, if required, be isolated from the cell culture by conventional separation techniques. Thus, for example, protein in the culture medium may be separated from cells by centrifugation, the supernatant containing the protein being collected after concentration (for example by ultrafiltration). The protein so obtained may be further purified, if desired, using conventional protein purification methods. Where the desired protein is not secreted into the culture medium during the culture it may obtained by rupture of the cells, then subsequent processing as just described.

(2) Advantages of the Invention Over Prior Approaches

Usefulness of the Present Invention

The advantage of adding a valproic acid compound to a culture system for the production of a protein of interest is that protein production can be substantially increased for a very small outlay in expenditure, and little if any alteration to culturing techniques. Moreover, the inventors show that adding a valproic acid compound enhances protein production more compared to adding sodium butyrate. Last, but not least, valproic acid is approved by the FDA for human usage, which makes it a safe compound to use for cell culture purposes.

Novelty of the Present Invention

As mentioned above, several patents have been filed around the use of alkanoic acids in general and sodium butyrate in particular in the context of enhanced production of a protein of interest. However, the use of a valproic acid compound in the context of enhancing production of a protein has not yet been published in prior art. And it cannot be assumed (without undue effort) that any alkanoic acid (including valproic acid) behaves similarly as sodium butyrate—unless one performs the necessary experiments. In particular, it cannot be assumed that adding a valproic acid increases batch yield more than adding sodium butyrate. The inventors—for the first time—show that adding a valproic acid compound enhances protein production more compared to adding sodium butyrate. This is also novel and has not been shown in prior art.

Non-Obviousness of the Present Invention

Whereas sodium butyrate has been extensively studied in the context of enhancing protein production, it is not obvious that all other alkanoic acids will have a similar effect. Moreover, the inventors show that valproic acid compounds enhance protein production more than sodium butyrate, which is also not obvious—unless one conducts the necessary experiments. Moreover, based on the use of valproic acid to treat seizures in humans, it seems quite a stretch to assume that it also has benefits in the production of recombinant proteins by mammalian cells in vitro.

Furthermore, given the high commercial interest in increasing batch yields and lowering production cost, and the ongoing research on the use of alkanoic acids to enhance protein production, the use of a valproic acid compound in that context would have been described already if it were obvious, particularly as valproic acid is an economically feasible alternative to sodium butyrate.

### DETAILED DESCRIPTION OF THE INVENTION

The practice of the present invention will employ, unless otherwise indicated, conventional methods of virology, microbiology, molecular biology and recombinant DNA techniques within the skill of the art. Such techniques are explained fully in the literature; see, e.g., Sambrook, et al. Molecular Cloning: A Laboratory Manual (Current Edition); DNA Cloning: A Practical Approach, vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, vol. I & II (P. Tijessen, ed.); Fundamental Virology, 2nd Edition, vol. I & II (B. N. Fields and D. M. Knipe, eds.)

(1) Definitions

In describing the present invention, the following terms will be employed, and are intended to be defined as indicated below.

For purposes of this invention, the term “enhancing agent” means a compound whose presence in a culture medium increases batch yield compared to the same process and culture medium in the absence of said compound.

For purposes of this invention, the term “valproic acid compound” or “VPA” means valproic acid, a valproic acid salt, a combination of valproic acid and valproic acid salt(s) or any other valproic acid derivative. Unless noted otherwise, VPA was purchased from Sigma-Aldrich Chemie GmbH, Industriestrasse 25, CH-9471 Buchs SG, Catalog Number P4543, and used in the experiments and embodiments of the present invention as described in the corresponding sections.

For purposes of this invention, the term “protein” means a polypeptide (native [i.e., naturally-occurring] or mutant), oligopeptide, peptide, or other amino acid sequence. As used herein, “protein” is not limited to native or full-length proteins, but is meant to encompass protein fragments having a desired activity or other desirable biological characteristics, as well as mutants or derivatives of such proteins or protein fragments that retain a desired activity or other biological characteristic including peptoids with nitrogen based backbone. Mutant proteins encompass proteins having an amino acid sequence that is altered relative to the native protein from which it is derived, where the alterations can include amino acid substitutions (conservative or non-conservative), deletions, or additions (e.g., as in a fusion protein). “Protein” and “polypeptide” are used interchangeably herein without intending to limit the scope of either term.

For purposes of this invention, “amino acid” refers to a monomeric unit of a peptide, polypeptide, or protein. There are twenty amino acids found in naturally occurring peptides, polypeptides and proteins, all of which are L-isomers. The term also includes analogs of the amino acids and D-isomers of the protein amino acids and their analogs.

For purposes of this invention, by “DNA” is meant a polymeric form of desoxyribonucleotides (adenine, guanine, thymine, or cytosine) in double-stranded or single-stranded form, either relaxed or supercoiled, either linear or circular. This term refers only to the primary and secondary structure of the molecule, and does not limit it to any particular tertiary forms. Thus, this term includes single- and double-stranded DNA found, inter alia, in linear DNA molecules (e.g., restriction fragments), viruses, plasmids, and chromosomes. In discussing the structure of particular DNA molecules, sequences may be described herein according to the normal convention of giving only the sequence in the 5′ to 3′ direction along the non-transcribed strand of DNA (i.e., the strand having the sequence homologous to the mRNA). The term captures molecules that include the four bases adenine (A or a), guanine (G or g), thymine (T or t), or cytosine (C or c), as well as molecules that include base analogues which are known in the art.

For purposes of this invention, “polynucleotide” as used herein means a polymeric form of nucleotides of any length, either ribonucleotides or desoxyribonucleotides. This term refers only to the primary structure of the molecule. Thus, the term includes double- and single-stranded DNA, as well as, double- and single-stranded RNA. It also includes modifications, such as methylation or capping, and unmodified forms of the polynucleotide.

For the purpose of describing the relative position of nucleotide sequences in a particular nucleic acid molecule throughout the instant application, such as when a particular nucleotide sequence is described as being situated “upstream,” “downstream,” “5′,” or “3′” relative to another sequence, it is to be understood that it is the position of the sequences in the non-transcribed strand of a DNA molecule that is being referred to as is conventional in the art.

For purposes of this invention, a “gene sequence” or “coding sequence” or “protein coding sequence” or “open reading frame” or “cDNA” or a sequence which “encodes” a particular protein, is a nucleic acid composition which is transcribed into RNA (in the case of DNA) and potentially translated (in the case of mRNA) into a polypeptide in vitro or in vivo when placed under the control of appropriate regulatory control elements. The boundaries of the gene are determined by a start codon at the 5′ (amino) terminus and potentially a translation stop codon at the 3′ (carboxy) terminus. A gene sequence can include, but is not limited to, cDNA from prokaryotic or eukaryotic mRNA, genomic DNA sequences from prokaryotic or eukaryotic DNA, and even synthetic DNA sequences. A transcription termination sequence, which is a particular species of regulatory control element, will usually be located 3′ to the protein coding sequence.

For purposes of this invention, by the term “transgene” is meant a nucleic acid composition made out of DNA, which encodes a peptide, oligopeptide or protein. The transgene may be operatively linked to regulatory control elements in a manner which permits transgene transcription, translation and/or ultimately directs expression of a product encoded by the expression cassette in the producer cell, e.g., the transgene is placed into operative association with a promoter and enhancer elements, as well as other regulatory control elements, such as introns or polyA sequences, useful for its regulation. The composite association of the transgene with its regulatory sequences (regulatory control elements) is referred to herein as a “minicassette”, “expression cassette”, “transgene expression cassette”, or “minigene”. The exact composition of the expression cassette will depend upon the use to which the resulting (mini)gene transfer vector will be put and is known to the artisan (Sambrook 1989, Lodish et al. 2000). When taken up by a target cell, the expression cassette as part of the recombinant vector genome may remain present in the cell as a functioning extrachromosomal molecule, or it may integrate into the cell's chromosomal DNA, depending on the kind of transfer vector used. Generally, a minigene may have a size in the range of several hundred base pairs up to about 30 kb.

For purposes of this invention, “heterologous” as it relates to nucleic acid compositions denotes sequences that are not normally joined together. Thus, a “heterologous” region of a nucleic acid composition is a segment of nucleic acid within or attached to another nucleic acid composition that is not found in association with the other molecule in nature. For example, a heterologous region of a nucleic acid composition could include a coding sequence flanked by sequences not found in association with the coding sequence in nature. Another example of a heterologous coding sequence is a construct where the coding sequence itself is not found in nature (e.g., synthetic sequences having codons different from the native gene). Allelic variation or naturally occurring mutational events do not give rise to heterologous DNA, as used herein.

For purposes of this invention, “homology” or “homologous” refers to the percent homology between two polynucleotide moieties or two polypeptide moieties. The correspondence between the sequence from one moiety to another can be determined by techniques known in the art. Two DNA or two polypeptide sequences are “substantially homologous” to each other when at least about 80%, preferably at least about 90%, and most preferably at least about 95% of the nucleotides or amino acids match over a defined length of the molecules, as determined using methods in the art.

The techniques for determining amino acid sequence homology are well-known in the art. In general, “homology” (for amino acid sequences) means the exact amino acid to amino acid comparison of two or more polypeptides at the appropriate place, where amino acids are identical or possess similar chemical and/or physical properties such as charge or hydrophobicity. A so-termed “percent homology” then can be determined between the compared polypeptide sequences. The programs available in the Wisconsin Sequence Analysis Package (available from Genetics Computer Group, Madison, Wis.), for example, the GAP program, are capable of calculating homologies between two polypeptide sequences. In addition, the ClustalW algorithm is capable of performing a similar analysis. Other programs and algorithms for determining homology between polypeptide sequences are known in the art.

Homology for polynucleotides is determined essentially as follows: Two polynucleotides are considered to be “substantially homologous” to each other when at least about 80%, preferably at least about 90%, and most preferably at least about 95% of the nucleotides match over a defined length of the molecules, when aligned using the default parameters of the search algorithm BLAST 2.0. The BLAST 2.0 program is publicly available. The ClustalW algorithm can be utilized as well.

Alternatively, homology for polynucleotides can be determined by hybridization experiments. As used herein, a nucleic acid sequence or fragment (such as for example, primers or probes), is considered to selectively hybridize to a sequence 1, thus indicating “substantial homology”, if such a sequence is capable of specifically hybridizing to the sequence 1 or a variant thereof or specifically priming a polymerase chain reaction: (i) under typical hybridization and wash conditions, such as those described, for example, in Maniatis, (Molecular Cloning: A Laboratory Manual, 2nd Edition, 1989) where preferred hybridization conditions are those of lesser stringency and more preferred, higher stringency; or (ii) using reduced stringency wash conditions that allow at most about 25-30% base pair mismatches, for example, 2.times.SSC, 0.1% SDS, at room temperature twice, for 30 minutes each; then 2×SSC, 0.1% SDS, 37° C., once for 30 minutes; the 2×SSC at room temperature twice, 10 minutes each or (iii) under standard PCR conditions or under “touch-down” PCR conditions.

For purposes of this invention, the term “cell” means any prokaryotic or eukaryotic cell, either ex vivo, in vitro or in vivo, either separate (in suspension) or as part of a higher structure such as but not limited to organs or tissues.

For purposes of this invention, the term “host cell” means a cell that can be transduced and/or transfected by an appropriate gene transfer vector. The nature of the host cell may vary from gene transfer vector to gene transfer vector.

For purposes of this invention, the term “producer cell” means a cell that is capable of producing a recombinant protein or protein of interest. The producer cell itself may be selected from any mammalian cell. Particularly desirable producer cells are selected from among any mammalian species, including, without limitation, cells such as HEK 293, A549, WEHI, 3T3, 10 T1/2, BHK, MDCK, COS 1, COS 7, BSC 1, BSC 40, BMT 10, VERO, WI38, HeLa, Saos, C2C12, L cells, HT1080, HepG2, CHO, NS0, Per.C6. The selection of the mammalian species providing the cells is not a limitation of this invention; nor is the type of mammalian cell, i.e., fibroblast, hepatocyte, tumor cell, etc. Frequently used producer cells or HEK 293 cells, BHK cells, NS0 cells, Per.C6 cells and CHO cells. Preferentially, a producer cell should be free of potential adventitious viruses.

For purposes of this invention, “transfection” is used to refer to the uptake of nucleic acid compositions by a cell. A cell has been “transfected” when an exogenous nucleic acid composition has crossed the cell membrane. A number of transfection techniques are generally known in the art. Such techniques can be used to introduce one or more nucleic acid compositions, such as a plasmid vector and other nucleic acid molecules, into suitable host cells. Frequently, cells are transfected with 25-kd linear polyethyleneimine. Other alternatives are transfection by means of electroporation, liposomes, dendrimers, or calcium phosphate.

For purposes of this invention, by “vector”, “transfer vector”, “gene transfer vector” or “nucleic acid composition transfer vector” is meant any element, such as a plasmid, phage, transposon, cosmid, chromosome, virus, virus capsid, virion, etc., which is capable of transferring and/or transporting a nucleic acid composition to a host cell, into a host cell and/or to a specific location and/or compartment within a host cell. Thus, the term includes cloning and expression vehicles, as well as viral and non-viral vectors and potentially naked or complexed DNA. However, the term does not include cells that produce gene transfer vectors such as retroviral packaging cell lines.

For purposes of this invention, the term “control elements”, “regulatory sequences” or “regulatory control elements” refers collectively to promoter regions, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control elements need always be present as long as the selected coding sequence is capable of being replicated, transcribed and/or translated in an appropriate host cell. Sometimes, the entirety of control elements and coding sequence is referred to as “gene”; in other instances, “gene” only refers to the coding sequence. For purposes of this invention, “gene” refers to the entirety of control elements and coding sequence. Expression control elements include appropriate transcription initiation, termination, promoter and enhancer sequences, efficient RNA processing signals such as splicing and polyadenylation signals, sequences that stabilize cytoplasmic mRNA, sequences that enhance translation efficacy (i.e., Kozak consensus sequence), sequences that enhance protein stability, and when desired, sequences that enhance protein processing and/or secretion. A great number of expression control elements, e.g., native, constitutive, inducible and/or tissue specific, are known in the art and may be utilized to drive expression of the gene, depending upon the type of expression desired. For eukaryotic cells, expression control elements typically include a promoter, an enhancer, such as one derived from an immunoglobulin gene, SV40, cytomegalovirus, etc., a polyadenylation sequence, and may include splice donor and acceptor sites. The polyadenylation sequence generally is inserted following the transgene sequences and before the 3′ ITR sequence in rAAV vectors.

The regulatory sequences useful in the constructs of the present invention may also contain an intron, desirably located between the promoter/enhancer sequence and the gene. One possible intron sequence is derived from SV40, and is referred to as the SV40 T intron sequence. Another suitable regulatory sequence includes the woodchuck hepatitis virus post-transcriptional element. Still other methods may involve the use of a second internal promoter, an alternative splice signal, a co- or post-translational proteolytic cleavage strategy, among others which are known to those of skill in the art. Selection of these and other common vector and regulatory sequences are conventional, and many such sequences are available. See, e.g., Sambrook et al, and references cited therein at, for example, pages 3.18-3.26 and 16.17-16.27 and Ausubel et al., CURRENT PROTOCOLS IN MOLECULAR BIOLOGY, John Wiley & Sons, New York, 1989.

One of skill in the art may make a selection among these regulatory sequences without departing from the scope of this invention. Suitable promoter/enhancer sequences may be selected by one of skill in the art using the guidance provided by this application. Such selection is a routine matter and is not a limitation of the present invention.

For purposes of this invention, the term “promoter” means a regulatory sequence capable of binding RNA polymerase and/or a regulatory sequence sufficient to direct transcription. “Promoter” is also meant to encompass those promoter (or enhancer) elements for cell-type specific, tissue-specific and/or inducible (by external signals or agents) transcription; such elements may be located in the 5′ or 3′ regions of a native gene.

For purposes of this invention, the term “operative association” or “operative linkage” refers to an arrangement of elements or nucleic acid sequences wherein the compounds so described are configured so as to perform their intended function. Thus, (a) regulatory sequence(s) operably linked to a coding sequence is/are capable of effecting the expression of said coding sequence and is/are connected in such a way as to permit gene expression of the coding sequence when the appropriate molecules (e.g., transcriptional activator proteins) are bound to the regulatory sequence(s). The regulatory sequences need not be contiguous with the coding sequence, as long as they function to direct the expression thereof. Thus, for example, intervening untranslated yet transcribed sequences can be present between a promoter sequence and the coding sequence and the promoter sequence can still be considered “operably linked” to the coding sequence. “Operably linked” sequences include both expression control sequences that are contiguous with the coding sequences for the product of interest and expression control sequences that act in trans or at a distance to control the expression of the product of interest.

For purpose of this invention, the term “specific productivity” refers to the amount of the protein of interest that is produced by a single cell per day. For example a specific productivity of 20 pg/cell/day refers to the production of 20 pg of the protein of interest by a single cell within 24 hours.

For purpose of this invention, the term “batch” refers to the (specific lot of) protein molecules of interest produced in a single production run, i.e., under the same production conditions. Batch means a specific quantity of a drug or other material that is intended to have uniform character and quality, within specified limits, and is produced according to a single manufacturing order during the same cycle of manufacture.

For purpose of this invention, the term “lot” means a batch, or a specific identified portion of a batch, having uniform character and quality within specified limits; or, in the case of a drug product produced by continuous process, it is a specific identified amount produced in a unit of time or quantity in a manner that assures its having uniform character and quality within specified limits

For purpose of this invention, the term “batch yield” refers to the maximum amount (in grams) of the recombinant protein of interest produced by all of the mammalian cells in the culture batch together. For secreted proteins, the “batch yield” refers to the maximum amount of the recombinant protein of interest in the culture medium where the recombinant protein of interest is secreted into the medium by the mammalian cells present in the medium. For example, if a mammalian cell culture of 1 liter comprises 0.5 g of recombinant protein of interest in total, the batch yield is 500 mg and the batch titer is 500 mg/l. Thus, whereas the specific productivity refers to the production of recombinant protein by a single mammalian cell within one day, the batch yield refers to the maximum amount of recombinant protein produced by all the mammalian cells in the culture during the total time of the culture. “Volumetric yield” can be used as a synonym for “batch yield”.

For purpose of this invention, the term “batch titer” refers to the maximum concentration (in grams per liter or milligrams per liter) of the recombinant protein of interest produced by all of the mammalian cells in the culture batch together. For secreted proteins, the “batch titer” refers to the maximum concentration of the recombinant protein of interest in the culture medium where the recombinant protein of interest is secreted into the medium by the mammalian cells present in the medium. For example, if a mammalian cell culture of 1 liter comprises 0.5 g of recombinant protein of interest in total, the batch yield is 0.5 grams and the batch titer is 0.5 g/l. Thus, whereas the specific productivity refers to the production of recombinant protein by a single mammalian cell within one day, the batch titer refers to the maximum concentration of recombinant protein produced by all the mammalian cells in the culture during the total time of the culture. The batch titer could also be defined as batch yield divided by culture volume.

For purpose of this invention, “growth medium” refers to a cell culture medium that promotes cell growth and division—leading to an increase in biomass as it relates to the cells. Optimally, a growth medium allows for a fast increase in biomass and supports cell growth to high cell densities.

For purpose of this invention, “transfection medium” refers to a cell culture medium that is suitable for transfection. Transfection media do not necessarily support cell growth or production. For example, RPMI can be used as transfection medium, but is not well suited for cell growth or production. An optimal transfection medium does not interfere with the transfection process, e.g., it does not contain inhibitors that inactivate the transfection reagent.

For purpose of this invention, “production medium” refers to a cell culture medium that promotes production of the protein of interest. A production medium does not necessarily support cell growth. Furthermore, one cannot necessarily transfect in production media, or only at a low transfection efficacy. An optimal production medium has the following characteristics: It sustains cell viability at a high cell density and results in high specific productivity for an extended period of time.

(2) General Methods

The practice of the present invention will employ, unless otherwise indicated, conventional methods of microbiology, molecular biology and recombinant DNA techniques within the skill of the art. Such techniques are explained fully in the literature; see, e.g., Sambrook, et al. Molecular Cloning: A Laboratory Manual (Current Edition); DNA Cloning: A Practical Approach, vol. I & II (D. Glover, ed.); Oligonucleotide Synthesis (N. Gait, ed., Current Edition); Nucleic Acid Hybridization (B. Hames & S. Higgins, eds., Current Edition); Transcription and Translation (B. Hames & S. Higgins, eds., Current Edition); CRC Handbook of Parvoviruses, vol. I & II (P. Tijessen, ed.); Fundamental Virology, 2nd Edition, vol. I & II (B. N. Fields and D. M. Knipe, eds.)

Unless otherwise noted, all experiments (examples) described in the following paragraphs were performed in triplicates.

### EXAMPLE 1

### Influence of VPA Addition on Batch Titers

10 million suspension-adapted HEK293E cells [1] were resuspended in 0.5 ml of Ex-Cell 293 HEK 293 serum-free medium with 4 mmol/l L-glutamine (Cat. No. 14571C-1000 M; Lot No. 6A0093; SAFC Biosciences, Lenexa, Kans., USA, “Ex-Cell” medium; note: “Ex-Cell” medium—as used herein—refers to a medium comprising 4 mmol/l L-glutamine) in a 50-ml filter tube each (TPP AG, Trasadingen, Switzerland; Cat. No. 87050, Lot 20050174). In a separate set, 10 million suspension-adapted CHO DG44 cells [6] were resuspended in 0.5 ml of ProCHO5 serum-free medium with 4 mmol/l L-glutamine and 0.68 g/l hypoxanthine and 0.194 g/l thymidine (BioWhittaker (Lonza), Belgium; Cat. No. BE12-766Q; Lot No. 7 MB0040; “ProCHO5” medium; note: “ProCHO5” medium—as used herein—refers to a medium comprising 4 mmol/l L-glutamine and 0.68 g/l hypoxanthine and 0.194 g/l thymidine) in a 50-ml filter tube each (TPP AG, Trasadingen, Switzerland; Cat. No. 87050, Lot 20050174).

Then 12.5 μg plasmid DNA at a concentration of 1 μg/μl was added in each tube with the following composition

50% (6.25 μg) p-LC (SEQ ID NO: 1)

50% (6.25 μg) p-HC (SEQ ID NO: 3).

Plasmid p-LC comprises the genetic information for the production of the light chain of an IgG antibody, plasmid p-HC comprises the genetic information for the production of the heavy chain of an IgG antibody. The particular vector constructs used in the present invention can also be obtained from the inventors. The inventors can be contacted via e-mail at hildinger@gmx.net. Moreover, commercial services do exist that produce any desired nucleotide sequence even comprising several kilobase pairs including complete expression cassettes in plasmid backgrounds (e.g., Invitrogen, Carlsbad, USA; Geneart, Germany.) Thus, providing the genetic sequence information of the plasmids should enable one of ordinary skill in the art to order the plasmids of the present invention at one of the commercial services listed.

After shortly mixing the DNA with the cells by gentle shaking, 25 μg of 25-kd linear polyethyleneimine (“PEI”; Polysciences, Eppelheim, Germany; [2]) were added at a concentration of 1 μg/μl to each of the tubes. After shortly mixing by gentle shaking, the filter tubes containing the cells together with the DNA and PEI were transferred into an orbital shaker (Kühner Shaker Cabinet ISF-4-W, “Kühner shaker”, Kühner AG, Birsfelden, Switzerland), and the cells were incubated for 4 hours at 37° C. in a 5% CO₂ atmosphere under shaking at 180 rpm. After that time, 4.5 ml of Ex-Cell medium were added to each of the filter tubes containing HEK 293 cells and 4.5 ml of Pro-CHO5 were added to each of the filter tubes containing CHO DG44 cells. Then, valproic acid was added to each of the tubes at the corresponding final concentration. The cells then were returned into the Kühner shaker and incubated at 37° C. in a 5% CO₂ atmosphere under shaking at 180 rpm. Five days post transfectionem, 200 μl of supernatant was removed from the cells in order to determine the antibody titer via ELISA.

The ELISA was performed as published in prior art [3]. In short, Goat anti-human kappa light chain IgG (Biosource) was used for coating the ELISA-plates, and with AP-conjugated goat anti-human gamma chain IgG (Biosource) the synthesized IgG1 was detected. NPP was used as a substrate for the alkaline phosphatase. Absorption was measured at 405 nm against 490 nm using a microplate reader (SPECTRAmax™340; Molecular Devices, Palo Alto, Calif., USA).

The following results were obtained at day 5 after transfection for HEK293E cells with standard deviation given as +/−values:

| Final VPA concentration | | Batch titer (mg/l) | Fold increase over control |
|---|---|---|---|
| 0 | mmol/l (control) | 15.0 ± 1.8 | 1 |
| 1.3 | mmol/l | 33.0 ± 4.3 | 2.2 |
| 2.5 | mmol/l | 61.8 ± 5.7 | 4.1 |
| 4 | mmol/l | 79.5 ± 3.6 | 5.3 |
| 5 | mmol/l | 64.5 ± 6.5 | 4.3 |
| 7.5 | mmol/l | 27.0 ± 3.1 | 1.8 |
| 11.3 | mmol/l | 13.5 ± 2.1 | 0.9 |

As one can see, batch titers are increasing when adding valproic acid. Only at very high valproic acid concentrations, batch titers are decreasing again due to lower cell viability. The optimal final concentration for HEK293E cells is at around 4 mmol/l.

The following results were obtained at day 5 after transfection for CHO cells with standard deviation given as +/−values:

| Final VPA concentration | | Batch titer (mg/l) | Fold increase over control |
|---|---|---|---|
| 0 | mmol/l (control) | 7.4 ± 1.2 | 1 |
| 0.05 | mmol/l | 7.4 ± 1.1 | 1 |
| 0.1 | mmol/l | 10.0 ± 1.5 | 1.3 |
| 0.2 | mmol/l | 13.7 ± 1.2 | 1.8 |
| 0.5 | mmol/l | 16.3 ± 1.3 | 2.1 |
| 1 | mmol/l | 14.4 ± 1.2 | 1.9 |
| 2 | mmol/l | 10.0 ± 0.9 | 1.3 |
| 3 | mmol/l | 7.4 ± 1.2 | 1.0 |

As one can see, batch titers are increasing when adding valproic acid. Only at very high valproic acid concentrations, batch titers are decreasing again due to lower cell viability. The optimal final concentration for CHO DG44 cells is at around 0.5 mmol/l.

### EXAMPLE 2

### Influence of Temperature on Batch Titer and Comparison with Sodium Butyrate

To analyze the influence of temperature on batch titer, 10 million suspension-adapted HEK293E cells [1] were resuspended in 0.5 ml of Ex-Cell 293 HEK 293 serum-free medium with 4 mmol/l L-glutamine (Cat. No. 14571C-1000M; Lot No. 6A0093; SAFC Biosciences, Lenexa, Kans., USA, “Ex-Cell” medium; note: “Ex-Cell” medium—as used herein—refers to a medium comprising 4 mmol/l L-glutamine) in a 50-ml filter tube each (TPP AG, Trasadingen, Switzerland; Cat. No. 87050, Lot 20050174). In a separate set, 10 million suspension-adapted CHO DG44 cells [6] were resuspended in 0.5 ml of ProCHO5 serum-free medium with 4 mmol/l L-glutamine and 0.68 g/l hypoxanthine and 0.194 g/l thymidine (BioWhittaker (Lonza), Belgium; Cat. No. BE12-766Q; Lot No. 7 MB0040; “ProCHO5” medium; note: “ProCHO5” medium—as used herein—refers to a medium comprising 4 mmol/l L-glutamine and 0.68 g/l hypoxanthine and 0.194 g/l thymidine) in a 50-ml filter tube each (TPP AG, Trasadingen, Switzerland; Cat. No. 87050, Lot 20050174).

Then 12.5 μg plasmid DNA at a concentration of 1 μg/μl was added in each tube with the following composition

50% (6.25 μg) p-LC (SEQ ID NO: 1)

50% (6.25 μg) p-HC (SEQ ID NO: 3).

Plasmid p-LC comprises the genetic information for the production of the light chain of an IgG antibody, plasmid p-HC comprises the genetic information for the production of the heavy chain of an IgG antibody. The particular vector constructs used in the present invention can also be obtained from the inventors. The inventors can be contacted via e-mail at hildinger@gmx.net. Moreover, commercial services do exist that produce any desired nucleotide sequence even comprising several kilobase pairs including complete expression cassettes in plasmid backgrounds (e.g., Invitrogen, Carlsbad, USA; Geneart, Germany.) Thus, providing the genetic sequence information of the plasmids should enable one of ordinary skill in the art to order the plasmids of the present invention at one of the commercial services listed.

After shortly mixing the DNA with the cells by gentle shaking, 25 μg of 25-kd linear polyethyleneimine (“PEI”; Polysciences, Eppelheim, Germany; [2]) were added at a concentration of 1 μg/μl to each of the tubes. After shortly mixing by gentle shaking, the filter tubes containing the cells together with the DNA and PEI were transferred into an orbital shaker (Kühner Shaker Cabinet ISF-4-W, “Kühner shaker”, Kühner AG, Birsfelden, Switzerland), and the cells were incubated for 4 hours at 37° C. in a 5% CO₂ atmosphere under shaking at 180 rpm. After that time, 4.5 ml of Ex-Cell medium were added to each of the filter tubes containing HEK 293 cells and 4.5 ml of Pro-CHO5 were added to each of the filter tubes containing CHO DG44 cells. Then, valproic acid was added to the corresponding tubes at the corresponding final concentration, and sodium butyrate (Cat. No. B5887-1G; Sigma-Aldrich Chemie GmbH, Industriestrasse 25, CH-9471 Buchs SG) was added to the corresponding tubes at the corresponding final concentrations. The cells then were returned into the Kühner shaker. Half the tubes were incubated at 37° C. in a 5% CO₂ atmosphere under shaking at 180 rpm, the other half was incubated at 31° C. Five days post transfectionem, 200 μl of supernatant was removed from the cells in order to determine the antibody titer via ELISA.

The ELISA was performed as published in prior art [3]. In short, Goat anti-human kappa light chain IgG (Biosource) was used for coating the ELISA-plates, and with AP-conjugated goat anti-human gamma chain IgG (Biosource) the synthesized IgG1 was detected. NPP was used as a substrate for the alkaline phosphatase. Absorption was measured at 405 nm against 490 nm using a microplate reader (SPECTRAmax™340; Molecular Devices, Palo Alto, Calif., USA).

The following results were obtained at day 5 after transfection for HEK293E cells with standard deviation given as +/−values:

| | Batch titer increase | Batch titer increase |
|---|---|---|
| Final concentration | at 31° C. | at 37° C. |
| Control (no VPA; no sodium | 1 | 1 |
| butyrate) | | |
| 3.8 mmol/l VPA | 5.3 ± 0.4 | 5.3 ± 0.5 |
| 3 mmol/l sodium butyrate | 4.8 ± 0.4 | 4.8 ± 0.4 |

As one can see, batch titers are increased both at 31° C. and 37° C., with VPA showing a slightly better performance than sodium butyrate at day 5 after transfection.

The following results were obtained at day 5 after transfection for CHO cells with standard deviation given as +/−values:

| | Batch titer increase | Batch titer increase |
|---|---|---|
| Final concentration | at 31° C. | at 37° C. |
| Control (no VPA; no sodium | 1 | 1 |
| butyrate) | | |
| 3.8 mmol/l VPA | 1.5 ± 0.1 | 2.1 ± 0.1 |
| 3 mmol/l sodium butyrate | 1.3 ± 0.1 | 1.9 ± 0.1 |

As one can see, batch titers are increased both at 31° C. and 37° C., with VPA showing a slightly better performance than sodium butyrate at day 5 after transfection.

### EXAMPLE 3

### Multiple VPA Additions

10 million suspension-adapted HEK293E cells [1] were resuspended in 0.5 ml of Ex-Cell 293 HEK 293 serum-free medium with 4 mmol/l L-glutamine (Cat. No. 14571C-1000M; Lot No. 6A0093; SAFC Biosciences, Lenexa, Kans., USA, “Ex-Cell” medium; note: “Ex-Cell” medium—as used herein—refers to a medium comprising 4 mmol/l L-glutamine) in a 50-ml filter tube each (TPP AG, Trasadingen, Switzerland; Cat. No. 87050, Lot 20050174). In a separate set, 10 million suspension-adapted CHO DG44 cells [6] were resuspended in 0.5 ml of ProCHO5 serum-free medium with 4 mmol/l L-glutamine and 0.68 g/l hypoxanthine and 0.194 g/l thymidine (BioWhittaker (Lonza), Belgium; Cat. No. BE12-766Q; Lot No. 7 MB0040; “ProCHO5” medium; note: “ProCHO5” medium—as used herein—refers to a medium comprising 4 mmol/l L-glutamine and 0.68 g/l hypoxanthine and 0.194 g/l thymidine) in a 50-ml filter tube each (TPP AG, Trasadingen, Switzerland; Cat. No. 87050, Lot 20050174).

Then 12.5 μg plasmid DNA at a concentration of 1 μg/μl was added in each tube with the following composition

50% (6.25 μg) p-LC (SEQ ID NO: 1)

50% (6.25 μg) p-HC (SEQ ID NO: 3).

Plasmid p-LC comprises the genetic information for the production of the light chain of an IgG antibody, plasmid p-HC comprises the genetic information for the production of the heavy chain of an IgG antibody. The particular vector constructs used in the present invention can also be obtained from the inventors. The inventors can be contacted via e-mail at hildinger@gmx.net. Moreover, commercial services do exist that produce any desired nucleotide sequence even comprising several kilobase pairs including complete expression cassettes in plasmid backgrounds (e.g., Invitrogen, Carlsbad, USA; Geneart, Germany.) Thus, providing the genetic sequence information of the plasmids should enable one of ordinary skill in the art to order the plasmids of the present invention at one of the commercial services listed.

After shortly mixing the DNA with the cells by gentle shaking, 25 μg of 25-kd linear polyethyleneimine (“PEI”; Polysciences, Eppelheim, Germany; [2]) were added at a concentration of 1 μg/μl to each of the tubes. After shortly mixing by gentle shaking, the filter tubes containing the cells together with the DNA and PEI were transferred into an orbital shaker (Kühner Shaker Cabinet ISF-4-W, “Kühner shaker”, Kühner AG, Birsfelden, Switzerland), and the cells were incubated for 4 hours at 37° C. in a 5% CO₂ atmosphere under shaking at 180 rpm. After that time, 4.5 ml of Ex-Cell medium were added to each of the filter tubes containing HEK 293 cells and 4.5 ml of Pro-CHO5 were added to each of the filter tubes containing CHO DG44 cells. Then, valproic acid was added to the corresponding tubes comprising HEK293E cells every other day (starting at day 0) to increase the final concentration from 1 mmol/l to 4 mmol/l in 1 mmol/l increments. Similarly, valproic acid was added to the corresponding tubes comprising CHO DG44 cells every other day (starting at day 0) to increase the final concentration from 0.125 mmol/l to 0.5 mmol/l in 0.125 mmol/l increments. The controls received 4 mmol/l valproic acid final concentration at day 0 (HEK293E) and 0.5 mmol/l (CHO DG44), respectively. The cells were incubated in a Kühner shaker at 37° C. in a 5% CO₂ atmosphere under shaking at 180 rpm. 10 days post transfectionem, 200 μl of supernatant was removed from the cells in order to determine the antibody titer via ELISA.

The ELISA was performed as published in prior art [3]. In short, Goat anti-human kappa light chain IgG (Biosource) was used for coating the ELISA-plates, and with AP-conjugated goat anti-human gamma chain IgG (Biosource) the synthesized IgG1 was detected. NPP was used as a substrate for the alkaline phosphatase. Absorption was measured at 405 nm against 490 nm using a microplate reader (SPECTRAmax™340; Molecular Devices, Palo Alto, Calif., USA).

The following results were obtained at day 10 after transfection for HEK293E cells:

| | | Standard |
|---|---|---|
| Final concentration | Batch titer (mg/l) | deviation (mg/l) |
| Control (4 mmol/l VPA at day 0) | 193 | 21 |
| VPA (escalating dosage) | 182 | 20 |

As one can see, batch titers are statistically not different if one adds VPA directly at the beginning or slowly increases the concentration over time. Yet, given that—from a process point of view—it will be easier to add VPA only once, direct addition of VPA to the final concentration should be preferred.

The following results were obtained at day 10 after transfection for CHO cells:

| Final concentration | Batch titer (mg/l) | Standard deviation (mg/l) |
|---|---|---|
| Control (0.5 mmol/l VPA at | 35.8 | 4.2 |
| day 0) | | |
| VPA (escalating dosage) | 37.9 | 3.1 |

As one can see, batch titers are statistically not different if one adds VPA directly at the beginning or slowly increases the concentration over time. Yet, given that—from a process point of view—it will be easier to add VPA only once, direct addition of VPA to the final concentration should be preferred.

### EXAMPLE 4

### Alternative Transgenes

10 million suspension-adapted HEK293E cells [1] were resuspended in 0.5 ml of Ex-Cell 293 HEK 293 serum-free medium with 4 mmol/l L-glutamine (Cat. No. 14571C-1000M; Lot No. 6A0093; SAFC Biosciences, Lenexa, Kans., USA, “Ex-Cell” medium; note: “Ex-Cell” medium—as used herein—refers to a medium comprising 4 mmol/l L-glutamine) in a 50-ml filter tube each (TPP AG, Trasadingen, Switzerland; Cat. No. 87050, Lot 20050174). In a separate set, 10 million suspension-adapted CHO DG44 cells [6] were resuspended in 0.5 ml of ProCHO5 serum-free medium with 4 mmol/l L-glutamine and 0.68 g/l hypoxanthine and 0.194 g/l thymidine (BioWhittaker (Lonza), Belgium; Cat. No. BE12-766Q; Lot No. 7MB0040; “ProCHO5” medium; note: “ProCHO5” medium—as used herein—refers to a medium comprising 4 mmol/l L-glutamine and 0.68 g/l hypoxanthine and 0.194 g/l thymidine) in a 50-ml filter tube each (TPP AG, Trasadingen, Switzerland; Cat. No. 87050, Lot 20050174).

Then 12.5 μg plasmid DNA at a concentration of 1 μg/μl was added in each tube with the following composition

100% (12.5 μg) p-TNFR-Fc (SEQ ID NO: 11).

Plasmid p-TNFR-Fc comprises the genetic information for the production of an Fc-tagged, soluble TNFα receptor. The particular vector construct used in the present invention can also be obtained from the inventors. The inventors can be contacted via e-mail at hildinger@gmx.net. Moreover, commercial services do exist that produce any desired nucleotide sequence even comprising several kilobase pairs including complete expression cassettes in plasmid backgrounds (e.g., Invitrogen, Carlsbad, USA; Geneart, Germany.) Thus, providing the genetic sequence information of the plasmids should enable one of ordinary skill in the art to order the plasmids of the present invention at one of the commercial services listed.

After shortly mixing the DNA with the cells by gentle shaking, 25 μg of 25-kd linear polyethyleneimine (“PEI”; Polysciences, Eppelheim, Germany; [2]) were added at a concentration of 1 μg/μl to each of the tubes. After shortly mixing by gentle shaking, the filter tubes containing the cells together with the DNA and PEI were transferred into an orbital shaker (Kühner Shaker Cabinet ISF-4-W, “Kühner shaker”, Kühner AG, Birsfelden, Switzerland), and the cells were incubated for 4 hours at 37° C. in a 5% CO₂ atmosphere under shaking at 180 rpm. After that time, 4.5 ml of Ex-Cell medium were added to each of the filter tubes containing HEK 293 cells and 4.5 ml of Pro-CHO5 were added to each of the filter tubes containing CHO DG44 cells. Then, valproic acid was added to each of the tubes at the corresponding final concentration. The cells then were returned into the Kühner shaker and incubated at 37° C. in a 5% CO₂ atmosphere under shaking at 180 rpm. Five days post transfectionem, 200 μl of supernatant was removed from the cells in order to determine the antibody titer via ELISA.

The ELISA was performed as published in prior art [3], but with a different coating antibody. In short, Goat Anti-Human IgG (Fc Fragment specific; Jackson ImmunoResearch Laboratories Inc., West Grove, Pa., USA; Code Number 109-006-098; Lot No. 72349) was used for coating the ELISA-plates, and with AP-conjugated goat anti-human gamma chain IgG (Biosource) the synthesized TNFR-Fc was detected. NPP was used as a substrate for the alkaline phosphatase. Absorption was measured at 405 nm against 490 nm using a microplate reader (SPECTRAmax™340; Molecular Devices, Palo Alto, Calif., USA).

The following results were obtained at day 5 after transfection for HEK293E cells with standard deviation given as +/−values:

| Final VPA concentration | Batch titer (mg/l) | Fold increase over control |
|---|---|---|
| 0 mmol/l (control) | 35.5 ± 2.8 | 1 |
| 3.8 mmol/l | 159.5 ± 12.0 | 4.5 |

As one can see, batch titers are increasing when adding valproic acid when using other (non-IgG) transgenes.

The following results were obtained at day 5 after transfection for CHO cells with standard deviation given as +/−values:

| Final VPA concentration | Batch titer (mg/l) | Fold increase over control |
|---|---|---|
| 0 mmol/l (control) | 15.4 ± 1.3 | 1 |
| 0.5 mmol/l | 27.7 ± 3.7 | 1.8 |

As one can see, batch titers are increasing when adding valproic acid when using other (non-IgG) transgenes.

### EXAMPLE 5

### Stable Cell Lines

20 million suspension-adapted HEK293E cells [1] were resuspended in 1 ml of Ex-Cell 293 HEK 293 serum-free medium with 4 mmol/l L-glutamine (Cat. No. 14571C-1000M; Lot No. 6A0093; SAFC Biosciences, Lenexa, Kans., USA, “Ex-Cell” medium; note: “Ex-Cell” medium—as used herein—refers to a medium comprising 4 mmol/l L-glutamine) in a 50-ml filter tube each (TPP AG, Trasadingen, Switzerland; Cat. No. 87050, Lot 20050174).

Then 50 μg plasmid DNA at a concentration of 1 μg/μl was added in each tube with the following composition

5% (2.5 μg) p-LC (SEQ ID NO: 1)

5% (2.5 μg) p-HC (SEQ ID NO: 3)

90% (45 μg) p-puro (SEQ ID NO: 13).

Plasmid p-LC comprises the genetic information for the production of the light chain of an IgG antibody, plasmid p-HC comprises the genetic information for the production of the heavy chain of an IgG antibody, plasmid p-puro comprises the genetic information for the enzyme puromycin-N-acetyl-transferase, which is encoded by the pac gene, the puromycin resistance gene. Expression of puromycin-N-acetyl-transferase confers resistance towards the antibiotic puromycin. The particular vector constructs used in the present invention can also be obtained from the inventors. The inventors can be contacted via e-mail at hildinger@gmx.net. Moreover, commercial services do exist that produce any desired nucleotide sequence even comprising several kilobase pairs including complete expression cassettes in plasmid backgrounds (e.g., Invitrogen, Carlsbad, USA; Geneart, Germany.) Thus, providing the genetic sequence information of the plasmids should enable one of ordinary skill in the art to order the plasmids of the present invention at one of the commercial services listed.

After shortly mixing the DNA with the cells by gentle shaking, 100 μg of 25-kd linear polyethyleneimine (“PEI”; Polysciences, Eppelheim, Germany [2]) were added at a concentration of 1 μg/μl to each of the tubes. After shortly mixing by gentle shaking, the filter tubes containing the cells together with the DNA and PEI were transferred into an orbital shaker (Kühner Shaker Cabinet ISF-4-W, “Kühner shaker”, Kühner AG, Birsfelden, Switzerland), and the cells were incubated for 3 hours at 37° C. in a 5% CO₂ atmosphere under shaking at 180 rpm. After that time, 4 ml of Ex-Cell medium were added to each of the filter tubes. One day after transfection, puromycin was added to a final concentration of 10 μg/ml (at this concentration of puromycin, only cells expressing the puromycin resistance gene can survive). The cells then were returned into the Kühner shaker and incubated at 37° C. in a 5% CO₂ atmosphere under shaking at 180 rpm.

One month after transfection, the surviving cells were pooled and tested for IgG expression to verify successful genomic integration of p-LC and p-HC by Southern blotting and PCR (data not shown). The stable mass culture was then split into two tube spins (10 ml each), and VPA was added to a final concentration of 4 mmol/l in one of the two tubes.

The same experiment was performed with CHO cells with the exception of using ProCHO5 as a medium and adding VPA to a final concentration of 0.5 mmol/l in one of the two tubes.

5 days after VPA addition, 200 μl of supernatant was removed from the cells in order to determine the antibody titer via ELISA.

The ELISA was performed as published in prior art [3]. In short, Goat anti-human kappa light chain IgG (Biosource) was used for coating the ELISA-plates, and with AP-conjugated goat anti-human gamma chain IgG (Biosource) the synthesized IgG1 was detected. NPP was used as a substrate for the alkaline phosphatase. Absorption was measured at 405 nm against 490 nm using a microplate reader (SPECTRAmax™340; Molecular Devices, Palo Alto, Calif., USA).

The following results were obtained at day 5 with standard deviation given as +/−values:

| | Batch titer | |
|---|---|---|
| Final VPA concentration | HEK293E (mg/l) | Batch titer CHO (mg/l) |
| 0 mmol/l (control) | 9.5 ± 1.1 | 12.8 ± 1.4 |
| 0.5 mmol/l | n/a | 35.5 ± 2.5 |
| 4 mmol/l | 47.8 ± 4.8 | n/a |

As one can see, VPA also increases batch titer for mammalian cells with stably integrated expression cassettes.

### EXAMPLE 6

### Combination with IDMTs

10 million suspension-adapted HEK293E cells [1] were resuspended in 0.5 ml of Ex-Cell 293 HEK 293 serum-free medium with 4 mmol/l L-glutamine (Cat. No. 14571C-1000M; Lot No. 6A0093; SAFC Biosciences, Lenexa, Kans., USA, “Ex-Cell” medium; note: “Ex-Cell” medium—as used herein—refers to a medium comprising 4 mmol/l L-glutamine) in a 50-ml filter tube each (TPP AG, Trasadingen, Switzerland; Cat. No. 87050, Lot 20050174). In a separate set, 10 million suspension-adapted CHO DG44 cells [6] were resuspended in 0.5 ml of ProCHO5 serum-free medium with 4 mmol/l L-glutamine and 0.68 g/l hypoxanthine and 0.194 g/l thymidine (BioWhittaker (Lonza), Belgium; Cat. No. BE12-766Q; Lot No. 7MB0040; “ProCHO5” medium; note: “ProCHO5” medium—as used herein—refers to a medium comprising 4 mmol/l L-glutamine and 0.68 g/l hypoxanthine and 0.194 g/l thymidine) in a 50-ml filter tube each (TPP AG, Trasadingen, Switzerland; Cat. No. 87050, Lot 20050174).

Then 12.5 μg plasmid DNA at a concentration of 1 μg/μl was added in each tube with the following composition

50% (6.25 μg) p-LC (SEQ ID NO: 1)

50% (6.25 μg) p-HC (SEQ ID NO: 3).

Plasmid p-LC comprises the genetic information for the production of the light chain of an IgG antibody, plasmid p-HC comprises the genetic information for the production of the heavy chain of an IgG antibody. The particular vector constructs used in the present invention can also be obtained from the inventors. The inventors can be contacted via e-mail at hildinger@gmx.net. Moreover, commercial services do exist that produce any desired nucleotide sequence even comprising several kilobase pairs including complete expression cassettes in plasmid backgrounds (e.g., Invitrogen, Carlsbad, USA; Geneart, Germany.) Thus, providing the genetic sequence information of the plasmids should enable one of ordinary skill in the art to order the plasmids of the present invention at one of the commercial services listed.

After shortly mixing the DNA with the cells by gentle shaking, 25 μg of 25-kd linear polyethyleneimine (“PEI”; Polysciences, Eppelheim, Germany; [2]) were added at a concentration of 1 μg/μl to each of the tubes. After shortly mixing by gentle shaking, the filter tubes containing the cells together with the DNA and PEI were transferred into an orbital shaker (Kühner Shaker Cabinet ISF-4-W, “Kühner shaker”, Kühner AG, Birsfelden, Switzerland), and the cells were incubated for 4 hours at 37° C. in a 5% CO₂ atmosphere under shaking at 180 rpm. After that time, 4.5 ml of Ex-Cell medium were added to each of the filter tubes containing HEK 293 cells and 4.5 ml of Pro-CHO5 were added to each of the filter tubes containing CHO DG44 cells. Then, valproic acid was added to each of the tubes at the corresponding final concentration. In addition, the following chemicals were added to the corresponding tubes at a final concentration of:

| Chemical added | | |
|---|---|---|
| (final concentration) | HEK-293E cells | CHO cells |
| VPA | 4 mmol/l | 0.5 mmol/l |
| Azacytidine | 6 μmol/l | 4 μmol/l |
| RG108 | 8 μmol/l | 60 μmol/l |

Azacytidine was purchased from Sigma-Aldrich Chemie GmbH, Industriestrasse 25, CH-9471 Buchs SG, Catalog Number A1287-1 VL. RG108 was purchased from Sigma-Aldrich Chemie GmbH, Industriestrasse 25, CH-9471 Buchs SG, Catalog Number R8279-10MG.

The cells then were returned into the Kuhner shaker and incubated at 37° C. in a 5% CO₂ atmosphere under shaking at 180 rpm. Five days post transfectionem, 200 μl of supernatant was removed from the cells in order to determine the antibody titer via ELISA.

The ELISA was performed as published in prior art [3]. In short, Goat anti-human kappa light chain IgG (Biosource) was used for coating the ELISA-plates, and with AP-conjugated goat anti-human gamma chain IgG (Biosource) the synthesized IgG1 was detected. NPP was used as a substrate for the alkaline phosphatase. Absorption was measured at 405 nm against 490 nm using a microplate reader (SPECTRAmax™340; Molecular Devices, Palo Alto, Calif., USA).

The following results were obtained at day 5 after transfection for HEK293E cells with standard deviation given as +/−values:

| Enhancer addition | Fold increase in batch yield |
|---|---|
| (final concentration) | over control |
| No addition | 100% |
| VPA | 520% ± 30% |
| VPA + Azacytidine | 510% ± 35% |
| VPA + RG108 | 525% ± 30% |
| Azacytidine | 160% ± 15% |
| RG108 | 150% ± 20% |

The following results were obtained at day 5 after transfection for CHO cells with standard deviation given as +/−values:

| Enhancer addition | Fold increase in batch yield |
|---|---|
| (final concentration) | over control |
| No addition | 100% |
| VPA | 170% ± 20% |
| VPA + Azacytidine | 350% ± 40% |
| VPA + RG108 | 340% ± 35% |
| Azacytidine | 160% ± 10% |
| RG108 | 170% ± 15% |

As one can see, Azacytidine and RG108 act synergistically together with VPA in increasing batch yield in the context of CHO cells.

### PREFERRED EMBODIMENT

20 million suspension-adapted HEK293E cells [1] were resuspended in 1 ml of Ex-Cell 293 HEK 293 serum-free medium with 4 mmol/l L-glutamine (Cat. No. 14571C-1000M; Lot No. 6A0093; SAFC Biosciences, Lenexa, Kans., USA, “Ex-Cell” medium; note: “Ex-Cell” medium—as used herein—refers to a medium comprising 4 mmol/l L-glutamine) in a 50-ml filter tube each (TPP AG, Trasadingen, Switzerland; Cat. No. 87050, Lot 20050174).

Then 50 μg plasmid DNA at a concentration of 1 μg/μl was added in each tube with the following composition

40% (20 μg) p-LC (SEQ ID NO: 1)

40% (20 μg) p-HC (SEQ ID NO: 3)

10% (5 μg) p-p18 (SEQ ID NO: 5)

10% (5 μg) p-p21 (SEQ ID NO: 7).

Plasmid p-LC comprises the genetic information for the production of the light chain of an IgG antibody, plasmid p-HC comprises the genetic information for the production of the heavy chain of an IgG antibody, plasmid p-p18 comprises the genetic information for the protein p18, and plasmid p-p21 comprises the genetic information for the protein p21. The particular vector constructs used in the present invention can also be obtained from the inventors. The inventors can be contacted via e-mail at hildinger@gmx.net. Moreover, commercial services do exist that produce any desired nucleotide sequence even comprising several kilobase pairs including complete expression cassettes in plasmid backgrounds (e.g., Invitrogen, Carlsbad, USA; Geneart, Germany.) Thus, providing the genetic sequence information of the plasmids should enable one of ordinary skill in the art to order the plasmids of the present invention at one of the commercial services listed.

After shortly mixing the DNA with the cells by gentle shaking, 100 μg of 25-kd linear polyethyleneimine (“PEI”; Polysciences, Eppelheim, Germany; [2]) were added at a concentration of 1 μg/μl to each of the tubes. After shortly mixing by gentle shaking, the filter tubes containing the cells together with the DNA and PEI were transferred into an orbital shaker (Kühner Shaker Cabinet ISF-4-W, “Kühner shaker”, Kühner AG, Birsfelden, Switzerland), and the cells were incubated for 3 hours at 37° C. in a 5% CO₂ atmosphere under shaking at 180 rpm. After that time, 4 ml of Ex-Cell medium were added to each of the filter tubes. Then, valproic acid or sodium butyrate (Cat. No. B5887-1G; Sigma-Aldrich Chemie GmbH, Industriestrasse 25, CH-9471 Buchs SG) was added to the corresponding tubes at the corresponding final concentrations. The cells then were returned into the Kühner shaker and incubated at 37° C. in a 5% CO₂ atmosphere under shaking at 180 rpm. 14 days post transfectionem, 200 μl of supernatant was removed from the cells in order to determine the antibody titer via ELISA.

The ELISA was performed as published in prior art [3]. In short, Goat anti-human kappa light chain IgG (Biosource) was used for coating the ELISA-plates, and with AP-conjugated goat anti-human gamma chain IgG (Biosource) the synthesized IgG1 was detected. NPP was used as a substrate for the alkaline phosphatase. Absorption was measured at 405 nm against 490 nm using a microplate reader (SPECTRAmax™340; Molecular Devices, Palo Alto, Calif., USA).

The following results were obtained at day 14 after transfection for HEK293E cells with standard deviation given as +/−values:

| | | Fold increase |
|---|---|---|
| Enhancing agent | Batch titer (mg/l) | over control |
| None | 73 ± 6.5 | 1 |
| 3.8 mmol/l VPA | 962 ± 73 | 13.2 |
| 3 mmol/l sodium butyrate | 528 ± 42 | 7.2 |

As one can see, batch titers are higher when adding VPA compared to adding sodium butyrate. (3 mmol/l sodium butyrate was determined to be the optimal concentration in combination with HEK293E cells). Based on the smaller difference between sodium butyrate and VPA at day 5 (see example 2), it seems that VPA exerts its effect at least in part by preserving viability/cellular productivity for a longer period of time.

### PRIOR ART CITATIONS

1. DUROCHER, Y, et al., A reporter gene assay for high-throughput screening of G-protein-coupled receptors stably or transiently expressed in HEK293 EBNA cells grown in suspension culture. Anal Biochem, 2000. 284(2): p. 316-26.
2. BALDI, L, et al., Transient gene expression in suspension HEK-293 cells: application to large-scale protein production. Biotechnol Prog, 2005. 21(1): p. 148-53.
3. MEISSNER, P, et al., Transient gene expression: recombinant protein production with suspension-adapted HEK293-EBNA cells. Biotechnol Bioeng, 2001. 75(2): p. 197-203.
4. STETTLER, M, et al., New disposable tubes for rapid and precise biomass assessment for suspension cultures of mammalian cells. Biotechnol Bioeng, 2006.
5. FUSSENEGGER, M, et al., Controlled proliferation by multigene metabolic engineering enhances the productivity of Chinese hamster ovary cells. Nat Biotechnol, 1998. 16(5): p. 468-72.
6. URLAUB, G, et al., Deletion of the diploid dihydrofolate reductase locus from cultured mammalian cells. Cell, 1983. 33(2):405-12
7. GALBRAITH, D. J., et al., Control of Culture Environment for Improved Polyethylenimine-Mediated Transient Production of Recombinant Monoclonal Antibodies by CHO Cells. Biotechnol. Prog. 2006, 22, 753-762
Some relevant prior art U.S. Pat. Nos. 5,681,718, 6,117,652, 6,740,505

What is claimed is:

1. A process for the production of a protein by a mammalian cell in culture, comprising: (a) providing a mammalian cell that produces a protein of interest, and (b) culturing said mammalian cell in a medium comprising a valproic acid compound selected from the group consisting of: (i) valproic acid, (ii) salts of valproic acid, and (iii) a combination of valproic acid and salts of valproic acid.

2. The process according to claim 1 wherein production of said protein in the presence of a valproic acid compound is enhanced compared to production of said protein in the absence of a valproic acid compound.

3. The process according to claim 1 wherein production of said protein in the presence of a valproic acid compound is enhanced compared to production of said protein in the presence of butyric acid or sodium butyrate.

4. The process according to claims 1 to 3 wherein said valproic acid compound is present at a concentration of between 0.002 mmol/l and 200 mmol/l, preferably between 0.01 mmol/l and 50 mmol/l and most preferably between 0.05 mM and 12 mmol/l.

5. The process of claims 1 to 3 where the mammalian cell is a HEK 293 cell and wherein said valproic acid compound is present at a concentration of 3.8 mmol/l.

6. The process of claims 1 to 3 where the mammalian cell is a CHO cell and wherein said valproic acid compound is present at a concentration of 0.5 mmol/l.

7. The process of claims 1 to 3, where the mammalian cell is a HEK 293 cell and wherein said valproic acid compound is present at a concentration of between 0.1 mmol/l and 12 mmol/l.

8. The process of claims 1 to 3, where the cell is a CHO cell and wherein said valproic acid compound is present at a concentration of between 0.05 mmol/l and 5 mmol/l.

9. The process of claims 1 to 8 where the valproic acid compound is added at a cell density of at least 1 million cells per ml.

10. The process of claims 1 to 8 where the valproic acid compound is added at a cell density of at least 2 million cells per ml.

11. The process of claims 1 to 8 where the valproic acid compound is added at a cell density of at least 4 million cells per ml.

12. The process of claims 1 to 11 where a valproic acid compound is added in combination with an inhibitor of DNA methyltransferases.

13. The process of claims 1 to 11 where a valproic acid compound is added in combination with a compound selected from the group consisting of azacytidine, RG108, decitabine.

14. The process of claims 1, 2, 3, 5, 6, 7, 8 where said mammalian cell is cultivated at a temperature of 37 degrees Celsius.

15. The process of claims 1, 2, 3, 5, 6, 7, 8 where said mammalian cell is cultivated at temperature of 31 degrees Celsius.

16. The process of claims 1, 2, 3, 5, 6, 7, 8 where said mammalian cell is cultivated at a temperature of between 28 degrees Celsius and 33 degrees Celsius.

17. The process according to claims 1 to 13 wherein said valproic acid compound is added at the beginning of the production phase.

18. The process according to claims 1 to 13 wherein said valproic acid compound is added at the beginning of the production phase and again at intervals thereafter.

19. The process according to claims 1, 2, 3, 5, 6, 7, 8, 14, 15, 16, comprising first culturing said eukaryotic cell in a cell culture medium that does not contain a valproic acid compound, prior to culturing said eukaryotic cell in a medium containing a valproic acid compound.

20. The process according to claims 1, 2, 3, 5, 6, 7, 8, 14, 15, 16, where said eukaryotic cell is cultured in a medium comprising a valproic acid compound for at least ten days.

21. A two stage process for the production of a protein according to claims 1, 2, 3 comprising: (a) a first stage of growing mammalian cells which produce said protein in growth medium until a predetermined cell density has been obtained; and (b) adding a valproic acid compound to said cells.

22. A three stage process for the production of a protein according to claims 1, 2, 3 comprising: (a) a first stage of expanding mammalian cells in growth medium; and (b) transfecting said mammalian cells with a gene expression vector encoding the protein of interest; and (c) cultivating said mammalian cells after transfection in a medium which contains a valproic acid compound.

23. The process of claims 1, 2, 3, 21, 22 where said protein is produced by means of transient gene expression.

24. The process of claims 1, 2, 3, 21 where said protein is produced by means of a stable producer cell.

25. The process of claim 23 where the valproic acid compound is added within 24 hours after transfection.

26. The process of claim 23 where the valproic acid compound is added within 48 hours after transfection.

27. The process according to claims 1, 2, 3, 5, 6, 7, 8, 14, 15, 16, 19, 20, 21, 22 wherein said eukaryotic cell is selected from mouse myeloma B (NS0) cells, Per.C6 cells, Chinese Hamster Ovary (CHO) cells, Baby Hamster Kidney (BHK) cells and Human Embryonic Kidney (HEK 293) cells.

28. The process of claims 23, 25, 26 where the transfected cell is a HEK 293 cell or CHO cell.

29. The process of claim 24 where the stable producer cell is a HEK 293 cell, CHO cell or Baby Hamster Kidney cell.

30. The process according to claims 1, 2, 3, 21, 22, 23, 24 wherein said protein is selected from the group consisting of growth factors, hormones, enzymes, enzyme inhibitors, cytokines, lymphokines, interleukins, blood clotting factors and immunoglobulins.

31. A process according to claims 1, 2, 3, 21, 22, 23, 24 wherein said protein is an immunoglobulin.

32. A process according to claim 1, 2, 3, 21, 22, 23, 24 wherein said protein is an Fc-fusion protein.
